(19) United States
(12) Patent Application Publication
(10) Pub. No.: US 2021/0375445 A1
(43) Pub. Date: Dec. 2, 2021

(54) Intraosseous Device and Data Transfer

(71) Applicant: Bard Access Systems, Inc., Salt Lake City, UT (US)

(72) Inventors: Eric W. Lindekugel, Salt Lake City, UT (US); Daniel B. Blanchard, Bountiful, UT (US)

(21) Appl. No.: 17/335,870

(22) Filed: Jun. 1, 2021

Related U.S. Application Data
(60) Provisional application No. 63/033,625, filed on Jun. 2, 2020.

(51) Int. Cl. G16H 40/40; H04W 4/38; G16H 40/67; A61M 39/02

(52) U.S. Cl. CPC G16H 40/40; H04W 4/38; G16H 40/67; A61M 39/0247; A61M 2039/025; A61M 2205/18; A61M 2205/8206; A61M 2039/0291; A61M 2205/52; A61M 2205/3523; A61M 2039/0258

(57) ABSTRACT

Disclosed herein are medical device systems, and methods thereof. Exemplary medical devices can include intraosseous (IO) access device systems, or similar medical devices that include a motive force. Embodiments include replaceable and rechargeable batteries that are configured to store one or more performance characteristics of the medical device and transfer the performance characteristic to a base station when the battery is removed from the medical device and coupled with the base station for recharging. The base station can then store performance characteristics from one or more batteries and/or medical devices. The performance characteristics can be transferred from the base station to one or more external computing devices or networks.

### PRIORITY

This application claims the benefit of priority to U.S. Provisional Application No. 63/033,625, filed Jun. 2, 2020, which is incorporated by reference in its entirety into this application.

### SUMMARY

Embodiments disclosed herein are directed to monitoring performance characteristics of medical device systems, and methods thereof. Exemplary medical device systems include intraosseous (IO) access devices including replaceable and rechargeable batteries. The battery can be configured to store one or more performance characteristics of the medical device and transfer the performance characteristic to a base station when the battery is removed from the medical device and coupled with the base station for recharging. The base station can then store and analyze the one or more performance characteristics from one or more batteries and/or medical devices. The performance characteristics can also be transferred from the base station to one or more external computing devices or networks. Similarly, information can be transferred to the medical device. Information such as system updates, software upgrades, patches, or the like, can be communicated to the base station and transferred to the medical device either by wired or wireless communication. In an embodiment, the information can be transferred, either wired or wirelessly from the base station to the battery and then transferred from the battery to the medical device when the recharged battery is disposed therein.

Typically, medical devices are required to be communicatively coupled with a computer that includes specific software in order to provide system updates and download performance characteristics for further analysis. This can interrupt the usage of the medical device and is often neglected due to the additional steps required, leading loss of valuable data as it is overwritten. Advantageously, transferring performance characteristics while recharging the battery integrates system updates and performance characteristic downloads with existing usage of the medical device. This provides a regular time interval between information transfer events, without having to interrupt the usage of the medical device itself, or losing performance data.

Disclosed herein is a medical device system including, a base station and a medical device including a battery including one of a processor, a persistent storage, or a communications logic, configured to store one of a performance characteristic or system update thereon, and configured to be removeable from the medical device and coupled with the base station to transfer the performance characteristic from the battery to the base station, or transfer the system update from the base station to the battery.

In some embodiments, the medical device system is an intraosseous access system and the medical device is a driver. The driver is configured to place an access assembly configured to access a vasculature of a patient. The access assembly includes one of a needle, a needle hub, an obturator, an obturator hub, or a safety shield. The obturator is disposed within a lumen of the needle and is configured to prevent a tissue from entering the needle lumen. The safety shield is configured to couple with a distal tip of the obturator as the obturator is removed from the needle lumen, to prevent accidental needle stick injuries. The base station is configured to recharge the removable battery when coupled thereto. The base station is communicatively coupled to one of an external computing device or a network.

In some embodiments, a performance characteristic includes one of make, model, or serial number of one of the medical device, the battery, or an access assembly, a number of placement events performed by the medical device, a number of attachments or detachments of the access assembly, a date or time that a placement event took place, a length of time the medical device was in use, a length of time a placement event took to complete, the length of time since a performance characteristic was last transferred to the base station, a torque of a motor, a speed of the motor, a number of rotations of the motor per placement, a charge level of the battery, a number of recharging events, a length of time since the battery was last recharged, an error in the hardware (e.g. the medical device, battery or base station), an error in the software (e.g. one or more logic), or an error in the operation of the medical device. The medical device further includes one of a second processor, a second persistent storage, or a second communications logic, configured to store a performance characteristic of the driver thereon, and configured to be coupled with one of the removable battery or the base station to transfer the performance characteristic from the medical device to one of the battery or the base station, or transfer the system update from one of the battery or the base station to the medical device.

In some embodiments, coupling the battery with the base station further includes one of wired communicative coupling or wireless communicative coupling, and wherein wireless communicative coupling includes one of Bluetooth, WiFi, Near Field Communication (NFC), or cellular Global System for Mobile Communication (GSM). In some embodiments, one of the base station or the medical device is configured to provide an alert to indicate to a user to couple one of the battery or the medical device with the base station to transfer the performance characteristic or system update therebetween. The alert is one of a visual, auditory, or tactile alert provided by one of the medical device, the battery or the base station. The battery includes a charge level indicator.

Also disclosed is a method of using a medical device system including, providing a base station, a medical device, and a removable battery configured to power the medical device, the removable battery including one of a processor, a storage, or a communications logic and configured to measure and store a performance characteristic of the medical device thereon, activating the medical device, measuring a performance characteristic of the medical device, storing the performance characteristic on the removable battery, removing the removable battery from the medical device, coupling the removable battery with the base station, and transferring the performance characteristic from the removable battery to the base station.

In some embodiments, the medical device system is an intraosseous access system and the medical device is a driver. Activating the medical device includes placing an access assembly to access a vasculature of a patient. The access assembly includes one of a needle, a needle hub, an obturator, an obturator hub, or a safety shield. The obturator is disposed within a lumen of the needle and is configured to prevent a tissue from entering the needle lumen. In some embodiments, the method further includes removing the obturator from the needle lumen after the needle has access the vasculature of the patient and wherein the safety shield is configured to couple with a distal tip of the obturator as the obturator is removed from the needle lumen, to prevent accidental needle stick injuries. In some embodiments, coupling the removable battery with the base station further includes recharging the removable battery. In some embodiments, the method further includes transferring the performance characteristic from the base station to one of an external computing device or a network.

In some embodiments, a performance characteristic includes one of make, model, or serial number of one of the medical device, the battery, or an access assembly, a number of placement events performed by the medical device, a number of attachments or detachments of the access assembly, a date or time that a placement event took place, a length of time the medical device was in use, a length of time a placement event took to complete, the length of time since a performance characteristic was last transferred to the base station, a torque of a motor, a speed of the motor, a number of rotations of the motor per placement, a charge level of the battery, a number of recharging events, a length of time since the battery was last recharged, an error in the hardware, an error in the software, or an error in the operation of the medical device. The medical device further includes one of a second processor, a second persistent storage, or a second communications logic, configured to measure and store a performance characteristic of the driver thereon.

In some embodiments, the medical device is communicatively coupled with the removable battery to transfer the performance characteristic from the medical device to the battery. Transferring the performance characteristic further includes one of a wired communicative coupling or a wireless communicative coupling, and wherein wireless communicative coupling includes one of Bluetooth, WiFi, Near Field Communication (NFC), or cellular Global System for Mobile Communication (GSM). In some embodiments, the method further includes providing an alert to indicate to a user to transfer the performance characteristic from one of the removable battery or the medical device to the base station. The alert is one of a visual, auditory, or tactile alert provided by one of the medical device, the removable battery or the base station.

Also disclosed is a method of updating a medical device system including, providing a base station, a medical device, and a removable battery configured to power the medical device, the removable battery including one of a processor, a storage, or a communications logic and configured to store a system update thereon, transferring the system update from an external computing device to the base station, removing the removable battery from the medical device, coupling the removable battery with the base station, transferring the system update from the base station to the removable battery, removing the removable battery from the base station, coupling the removable battery with the medical device, and transferring the system update from the removable battery to the medical device.

In some embodiments, the medical device system is an intraosseous access system and the medical device is a driver. Coupling the removable battery with the base station further includes recharging the removable battery. One of i) transferring the system update from an external computing device to the base station, ii) transferring the system update from the base station to the removable battery, or iii) transferring the system update from the removable battery to the medical device, includes one of a wired communicative coupling or a wireless communicative coupling, and wherein wireless communicative coupling includes one of Bluetooth, WiFi, Near Field Communication (NFC), or cellular Global System for Mobile Communication (GSM). In some embodiments, the method further includes providing an alert to indicate to a user to transfer the system update to the medical device. The alert is one of a visual, auditory, or tactile alert provided by one of the medical device, the removable battery, or the base station.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates an exploded view of an embodiment of an intraosseous access medical device system, wherein an access assembly subset of the system is depicted slightly enlarged and in elevation, and an automated driver component is depicted in perspective, in accordance with embodiments disclosed herein.

FIG. 2A illustrates a side view of an intraosseous access medical device system, in accordance with embodiments disclosed herein.

FIG. 2B illustrates a cross-sectional view of an intraosseous access medical device system, in accordance with embodiments disclosed herein.

FIG. 3 illustrates a schematic view of an exemplary environment of use for an intraosseous access medical device system, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

### Terminology

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a needle disclosed herein includes a portion of the needle intended to be near a clinician when the needle is used on a patient. Likewise, a “proximal length” of, for example, the needle includes a length of the needle intended to be near the clinician when the needle is used on the patient. A “proximal end” of, for example, the needle includes an end of the needle intended to be near the clinician when the needle is used on the patient. The proximal portion, the proximal end portion, or the proximal length of the needle can include the proximal end of the needle; however, the proximal portion, the proximal end portion, or the proximal length of the needle need not include the proximal end of the needle. That is, unless context suggests otherwise, the proximal portion, the proximal end portion, or the proximal length of the needle is not a terminal portion or terminal length of the needle.

With respect to “distal,” a “distal portion” or a “distal end portion” of, for example, a needle disclosed herein includes a portion of the needle intended to be near or in a patient when the needle is used on the patient. Likewise, a “distal length” of, for example, the needle includes a length of the needle intended to be near or in the patient when the needle is used on the patient. A “distal end” of, for example, the needle includes an end of the needle intended to be near or in the patient when the needle is used on the patient. The distal portion, the distal end portion, or the distal length of the needle can include the distal end of the needle; however, the distal portion, the distal end portion, or the distal length of the needle need not include the distal end of the needle. That is, unless context suggests otherwise, the distal portion, the distal end portion, or the distal length of the needle is not a terminal portion or terminal length of the needle.

In the following description, certain terminology is used to describe aspects of the invention. For example, in certain situations, the term “logic” is representative of hardware, firmware or software that is configured to perform one or more functions. As hardware, logic may include circuitry having data processing or storage functionality. Examples of such circuitry may include, but are not limited or restricted to a hardware processor (e.g., microprocessor with one or more processor cores, a digital signal processor, a programmable gate array, a microcontroller, an application specific integrated circuit “ASIC,” etc.), a semiconductor memory, or combinatorial elements.

Alternatively, logic may be software, such as executable code in the form of an executable application, an Application Programming Interface (API), a subroutine, a function, a procedure, an applet, a servlet, a routine, source code, object code, a shared library/dynamic load library, or one or more instructions. The software may be stored in any type of a suitable non-transitory storage medium, or transitory storage medium (e.g., electrical, optical, acoustical or other form of propagated signals such as carrier waves, infrared signals, or digital signals). Examples of non-transitory storage medium may include, but are not limited or restricted to a programmable circuit; semiconductor memory; non-persistent storage such as volatile memory (e.g., any type of random access memory “RAM”); or persistent storage such as non-volatile memory (e.g., read-only memory “ROM,” power-backed RAM, flash memory, phase-change memory, etc.), a solid-state drive, hard disk drive, an optical disc drive, or a portable memory device. As firmware, the executable code may be stored in persistent storage.

The term “computing device” should be construed as electronics with the data processing capability and/or a capability of connecting to any type of network, such as a public network (e.g., Internet), a private network (e.g., a wireless data telecommunication network, a local area network “LAN”, etc.), or a combination of networks. Examples of a computing device may include, but are not limited or restricted to, the following: a server, an endpoint device (e.g., a laptop, a smartphone, a tablet, a “wearable” device such as a smart watch, augmented or virtual reality viewer, or the like, a desktop computer, a netbook, a medical device, or any general-purpose or special-purpose, user-controlled electronic device), a mainframe, internet server, a router; or the like.

A “message” generally refers to information transmitted in one or more electrical signals that collectively represent electrically stored data in a prescribed format. Each message may be in the form of one or more packets, frames, HTTP-based transmissions, or any other series of bits having the prescribed format.

The term “computerized” generally represents that any corresponding operations are conducted by hardware in combination with software and/or firmware.

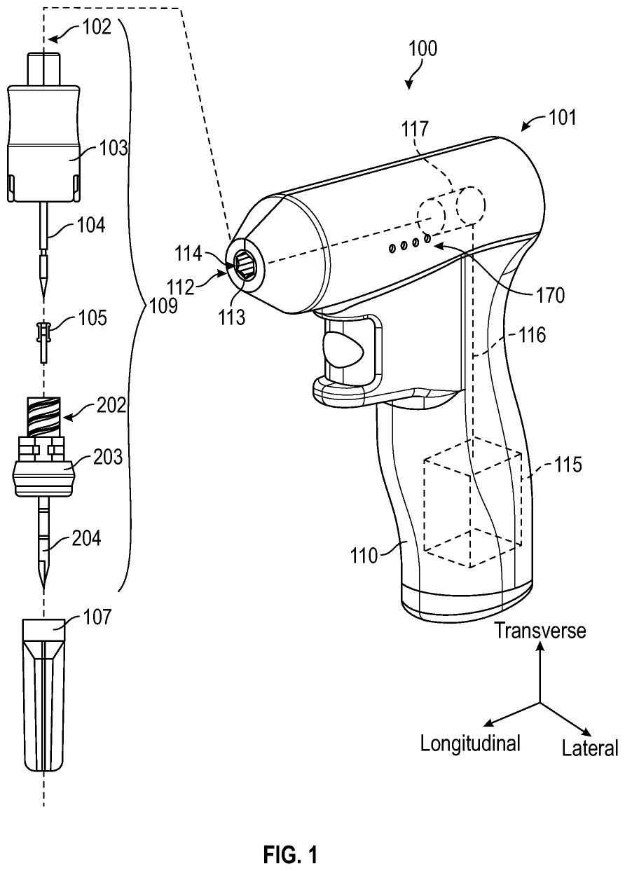

As shown in FIG. 1, and to assist in the description of embodiments described herein, a longitudinal axis extends substantially parallel to an axial length of a needle 204 extending from the driver 101. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

The present disclosure relates generally to monitoring performance characteristics of medical devices and methods thereof. Exemplary medical devices can include intraosseous (IO) access device systems, ultrasound systems, medical device tracking systems, or similar electronic devices that can include a rechargeable and/or replaceable battery. FIG. 1 shows an exploded view of an exemplary intraosseous access system (“system”) 100, with some components thereof shown in elevation and another shown in perspective. However, it will be appreciated that this medical device is exemplary and not intended to be limiting in any way. In an embodiment, the intraosseous access system 100 can be used to penetrate skin and underlying hard bone (“bone cortex”) for intraosseous access, such as, for example to access the marrow of the bone and/or a vasculature of the patient via a pathway through an interior of the bone (“medullary cavity”).

In an embodiment, the system 100 includes a driver 101 and an access assembly 109. The driver 101 can be used to rotate the access assembly 109 and “drill” a needle 204 into the bone of a patient. In embodiments, the driver 101 can be automated or manual. As shown, the driver 101 is an automated driver 101. For example, the automated driver 101 can be a drill that achieves high rotational speeds. In an embodiment, the intraosseous access system 100 can further include an obturator assembly 102, a safety shield (“shield”) 105, and a needle assembly 202, which may be referred to, collectively, as the access assembly 109. The needle assembly 202 can include an access needle (“needle”) 204 supported by a needle hub 203. In an embodiment, the obturator assembly 102 includes an obturator 104, configured to prevent bone fragments, tissue, or the like from entering the needle lumen and obstructing a fluid flow therethrough after the needle has been placed. As will be appreciated, in some embodiments, the obturator 104 may be replaced with a different elongated medical instrument. As used herein, the term “elongated medical instrument” is a broad term used in its ordinary sense that includes, for example, such devices as needles, cannulas, trocars, obturators, stylets, and the like. Accordingly, the obturator assembly 102 may be referred to more generally as an elongated medical instrument assembly. In like manner, the obturator 104 may be referred to more generally as an elongated medical instrument.

In an embodiment, the obturator assembly 102 includes a coupling hub 103 that is attached to the obturator 104 in any suitable manner (e.g., one or more adhesives or overmolding, etc.). The coupling hub 103 can be configured to interface with the driver 101. The coupling hub 103 may alternatively be referred to as an obturator hub 103 or, more generally, as an elongated instrument hub 103. In an embodiment, the shield 105 is configured to couple with the obturator 104 to prevent accidental needle stick injuries when the obturator is removed after placement of the needle 204.

In an embodiment, the needle assembly 202 includes a needle 204. However, in some embodiments, the needle 204 may be replaced with a different instrument, such as, for example, a cannula, a tube, or a sheath, and/or may be referred to by a different name, such as one or more of the foregoing examples. Accordingly, the needle assembly 202 may be referred to more generally as a cannula assembly or as a tube assembly. In like manner, the needle 204 may be referred to more generally as a cannula.

In an embodiment, the needle assembly 202 includes a needle hub 203 that is attached to the needle 204 in any suitable manner. The needle hub 203 can be configured to couple with the obturator hub 103 and may thereby be coupled with the driver 101, as further discussed below. The needle hub 203 may alternatively be referred to as a cannula hub 203. In an embodiment, a cap 107 may be provided to cover at least a distal portion of the needle 204 and the obturator 104 prior to use of the access assembly 109. For example, in an embodiment, a proximal end of the cap 107 can be coupled to the obturator hub 103.

With continued reference to FIG. 1, the driver 101 may take any suitable form. The driver 101 may include a handle 110 that may be gripped by a single hand of a user. In an embodiment, the driver 101 further includes a coupling interface 112, which is formed as a socket 113 that defines a cavity 114. The coupling interface 112 can be configured to couple with the obturator hub 103. In an embodiment, the socket 113 includes sidewalls that substantially define a hexagonal cavity into which a hexagonal protrusion of the obturator hub 103 can be received. Other suitable connection interfaces are also contemplated.

The driver 101 can include an energy source 115 of any suitable variety that is configured to energize the rotational movement of the coupling interface 112 and provide a motive force. For example, in some embodiments, the energy source 115 may comprise one or more batteries that provide electrical power for the driver 101. In some embodiments, the energy source 115 can comprise one or more springs (e.g., a coiled spring, flat spring, or the like) or other biasing member that may store potential mechanical energy that may be released upon actuation of the driver 101.

The energy source 115 may be coupled with the coupling interface 112 in any suitable manner. For example, in an embodiment, the driver 101 includes an electrical, mechanical, or electromechanical coupling 116 to a gear assembly 117. In some embodiments, the coupling 116 may include an electrical motor that generates mechanical movement from electrical energy provided by an electrical energy source 115. In other embodiments, the coupling 116 may include a mechanical linkage to the gear assembly 117. The driver 101 can include a mechanical coupling of any suitable variety to couple the gear assembly 117 with the coupling interface 112. In other embodiments, the gear assembly 117 may be omitted.

Further details and embodiments of the intraosseous access system 100 can be found in WO 2018/075694, WO 2018/165334, WO 2018/165339, US 2021/0093358 and U.S. Pat. No. 10,893,887, each of which are incorporated by reference in their entirety into this application.

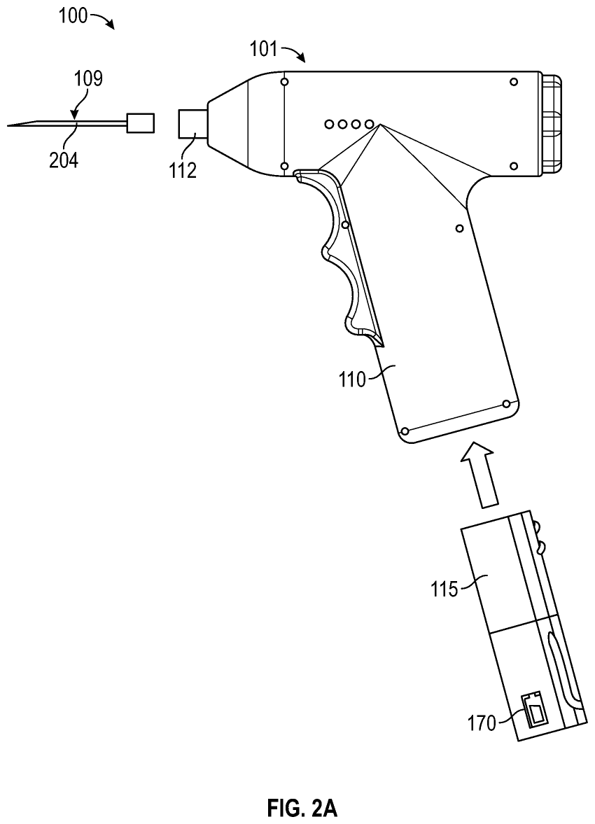

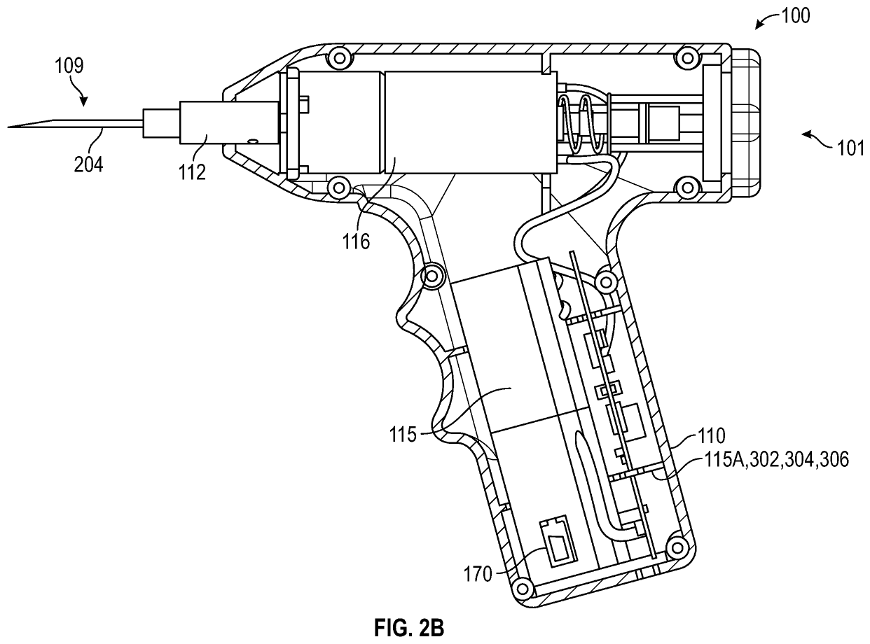

FIG. 2A shows an embodiment of an intraosseous access device 100, including a driver 101 that includes a rechargeable, replaceable, battery pack energy source (“battery pack”) 115. In an embodiment the battery pack 115 is removable and replaceable with similar battery packs. Advantageously, this allows a user of the system 100 to ensure there is sufficient power when the system 100 is deployed in a placement event. Further, during a placement event, should the power be depleted from the first battery pack, a user can replace the first battery pack with a second, fully charged battery pack and continue the access procedure without having to wait for the first battery pack to be recharged. As used herein, a placement event is considered an event where the medical device system 100 carries out its intended purpose, for example the intraosseous access system 100 placing a needle 204 to access an intramedullary cavity of a bone.

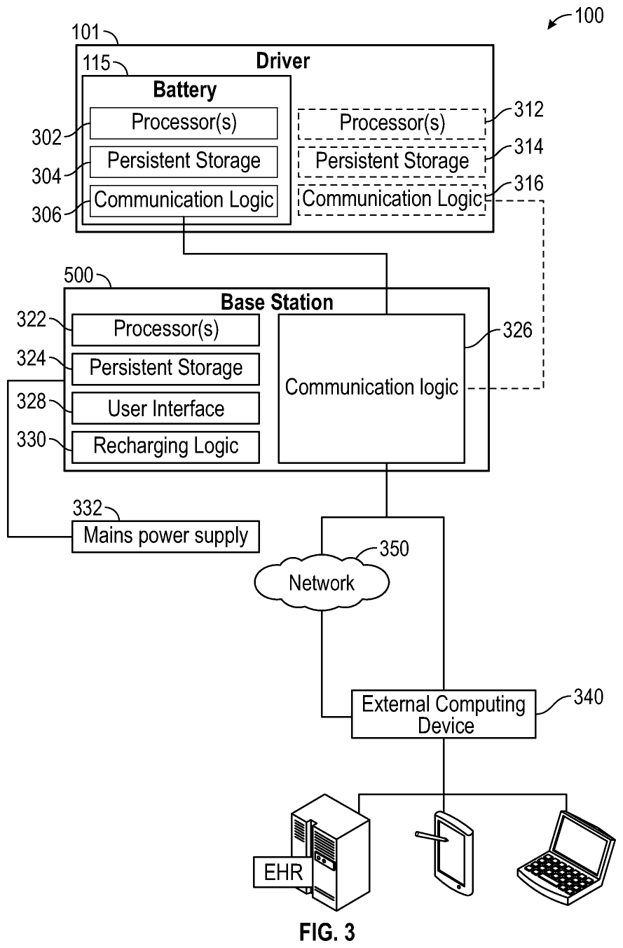

FIG. 2B shows a cross-sectional view of the intraosseous device 100 of FIG. 2A with the rechargeable battery pack 115 disposed therein. FIG. 3 shows a schematic view of the access system 100 in an exemplary environment of use. In an embodiment, the battery pack 115 can include a logic board 115A that can include one of a processor(s) 302, storage 304 (i.e. non-transitory storage medium or transitory storage medium), or communications logic 306, configured to record and store a performance characteristic or associated parameters of the system 100.

Exemplary performance characteristics can include, but not limited to, information about the medical device system 100 for example, make, model, serial number of the medical device, e.g. driver 101, battery 115, access assembly 109, or components thereof, information relating the use of the medical device, for example the number of placements events, the number of attachments or detachments of the access assembly, the date/time the placement took place, the length of time the medical device was in use, the length of time for a placement event to complete, the length of time since a performance characteristic was last transferred to the base station, the length of time since a system update was last transferred to the medical device, information on the operation of the medical device, e.g. torque (lbs/ft), speed (rpm), and number of rotations per placement event, operation time, battery charge level (volts), number of recharging events, health or lifespan of the battery or components of the medical device, a length of time since the battery was last recharged, any errors or anomalies recorded in the hardware, software, or operation of the device, combinations thereof, or the like. In an embodiment, performance characteristics can include any information relating to the operation of the device, information relating to the interaction of the device, or any other relevant information. For example, where the medical device is a tracking device, the information can relate to a tracking event rather than a placement event.

In an embodiment, the medical device itself, e.g. driver 101, can include one of processor 302, storage 304, or communications logic 306 configured to record and store information about the performance of the system 100, as described herein. In an embodiment, one or more of the processor 302, storage 304, or communications logic 306 can be disposed on either the access device 101, or the battery 115 and can be communicatively coupled therebetween. In an embodiment, the battery 115 can include one of a first processor 302, a first storage 304, or a first communications logic 306, which can be communicatively coupled with one of a second processor 312, a second storage 314, or a second communications logic 316 disposed on the driver 101.

In an embodiment, one of the driver 101, battery 115, or combinations thereof can include a battery charge indicator 170. The battery charge indicator 170 can include one or more LED lights, LCD display, icons, dial indicators, gauges, auditory, visual, or tactile indicators, or the like, which can turn on or off, change color, or combinations thereof, to indicate a level of charge of the battery pack 115. In an embodiment, the system 100 includes a charge indicator button that a user can actuate to activate the battery charge indicator 170 and determine a charge level for the battery pack 115.

In an embodiment, the intraosseous access system 100 can further include a base station 500. The base station 500 can include one of a third processor 322, a third storage 324, or a third communications logic 326. The base station 500 can further include a user interface 328 or a recharging logic 330. The recharging logic 330 can be configured to recharge the battery 115 from an external mains power supply 332. The user interface 180 can include one or more physical push buttons, switches, dials, sliders, screens, touchscreens, lights, LED lights, speakers, combinations or the like configured to display information and receive inputs from a user.

In an embodiment, the battery 115 and associated first processor 302, first storage 304, or first communications logic 306, or the like, can be removed from the medical device, e.g. driver 101 and can be coupled with the base station 500 to recharge the battery 115. In an embodiment, the driver 101, with the battery 115 disposed therein, can be coupled with the base station 500 and can recharge the battery 115.

In an embodiment, the battery 115 can be communicatively coupled, either wired or wirelessly, with the base station 500 and can transfer one or more performance characteristics to the base station 500. In an embodiment, the base station 500 can transfer one or more system updates to the battery 115 that can then be transferred to the medical device, e.g. driver 101, when the recharged battery 115 is replaced in the medical device. Exemplary wireless communication can include Bluetooth, WiFi, Near Field Communication (NFC), cellular Global System for Mobile Communication (GSM), combinations thereof, or the like.

In an embodiment, the base station 500 can be coupled, either wired or wirelessly, with one or more external computing devices 340 or networks 350. Exemplary external devices external computing devices 340 or networks 350 can include external monitors, laptop, computers, mobile devices, smart phones, tablets, “wearable” electronic devices, servers, centralized or decentralized networks, hospital intranet server, Electronic Health Record (“EHR”) systems, “cloud” based networks, internet, combinations thereof, or the like.

In an embodiment, the one or more performance characteristics of the system 100 can be stored on the driver 101, battery 115, or combinations thereof, and can be transferred to the base station 500. The base station 500 can then store and analyze the one or more performance characteristics and/or transfer the one or more performance characteristic to one or more external computing devices 340 or networks 350. Similarly, system updates or the like can be transferred from the one or more external computing devices 340 to the base station 500. The base station 500 can then transfer the system updates either directly to the medical device, e.g. driver 101, or to the battery 115. The battery 115 can then transfer the system update, or similar information, to the medical device when it is coupled thereto.

Advantageously, performance characteristics can be communicated and stored to the base station 500 at a regular time interval or in response to a trigger. For example, the trigger can be a time based trigger or an action based trigger. A performance characteristic can be communicated and stored to the base station 500 after a predetermined time period has elapsed, or in response to a predetermined action. Exemplary action triggers can include when the battery 115 needs recharging, after an access event, one of the base station 500, driver 101, or battery 115 being disconnected or connected, in response to an input from a user provided to one of the base station 500, driver 101, or battery 115, in response to an input from one of the base station 500, driver 101, or battery 115 (e.g. a fault has been detected), or in response to an input from an external computing device 340, or the like. The performance information can then be communicated with one the external computing device(s) 340 to be analyzed and to monitor the performance of the battery 115, driver 101, or system 100. For example, a hospital or similar institution can monitor when an access system 100 was last used, when the system 100 needs to be replaced, which devices may be faulty or need attention, or the like. Similarly, manufacturers can provide system updates, or monitor usage or performance of one or more intraosseous access systems 100 or similar medical device systems, in order to improve future devices or provide updates to existing systems.

In an embodiment, one of the driver 101, battery 115, base station 500, or combinations thereof can provide an alert to the user to indicate that the battery 115 needs to be removed from the driver 101 and coupled with a base station 500. The alert can include a visual, auditory, or tactile indication to alert a user that the battery 115 needs to be recharged or that information on the performance characteristics needs to be transferred to the base station 500, e.g. either because a predetermined time period has elapsed, because the storage 304 is nearing capacity, or the like.

In an embodiment, the energy source 115 of the medical device, e.g. for placing the access assembly 109, can include a spring or similar biasing member. The spring driven driver 101 can include a processor 312, storage 314, communication logic 316, additional power source (e.g. battery), combinations thereof or the like, and can be configured to measure and store the performance information of the spring driven driver 101. The driver 101 can then communicate the information with the base station 500 and transfer the information to one or more external computing devices, as described herein.

In an exemplary method of use a medical device system (e.g. an intraosseous access system 100) can be provided including a medical device (e.g. driver 101), battery 115, and a base station 500. One of the medical device (e.g. driver 101) or battery 115 can include one of a processor 302, storage 304, or communications logic 306, or combinations thereof, configured to measure and store a performance characteristic of the medical device. In an embodiment, the performance characteristic can be stored on the battery 115. In an embodiment, the performance characteristic can be stored on the driver 101 and transferred to the battery 115, which can then be transferred to the base station 500. In an embodiment, the performance characteristic can be stored on the driver 101 and transferred directly to the base station 500.

In an embodiment, the performance characteristic can be transferred to the battery 115. The battery 115 can then be removed from the driver 101 and coupled with the base station 500. Optionally, a second battery can be disposed within the driver 101 and can continue to power the driver 101 and record the performance characteristics. In an embodiment, the battery 115 can be coupled with the recharging logic 330 and can recharge the battery 115. In an embodiment, the battery 115 can be coupled, either wired or wirelessly, with the base station communication logic 326 to transfer performance characteristics from the battery 115 to the base station 500.

In an embodiment, a driver 101 with the battery 115 disposed therein can be coupled with the recharging logic 330 and can recharge the battery 115. In an embodiment, the driver 101 can be coupled, either wired or wirelessly, with the base station communication logic 326 to transfer performance characteristics from one of the driver 101 or the battery 115 to the base station 500. In an embodiment, the base station 500 can store information from one or more battery(s) 115 and can transfer the performance characteristics, either wired or wirelessly, to one or more networks 350 or external computing devices 340.

In an embodiment, a system update can be transferred from the one or more external computing devices 340 to the base station 500 either directly, or by way of a network 350. The base station 500 can be configured to transfer the system update to one of the battery 115, or driver 101, when coupled thereto. In an embodiment, when the battery 115 has been recharged, the battery 115 can then be removed from the base station 500 and coupled with the driver 101 to transfer the system update to the medical device and continue to measure a performance characteristic. In an embodiment, the medical device, e.g. driver 101, with the battery 115 disposed therein, can be coupled with the base station 500. The base station 500 can be configured to transfer the system update to the driver 101 directly.

As will be appreciated although embodiments are described herein in terms of performance characteristics of an intra-osseous access system 100, embodiments can also be used with various electronic medical devices, e.g. ultrasound systems, medical device tracking systems, or similar electronic devices. Accordingly, performance characteristics of the medical device can be stored to a removable, rechargeable battery 115 and transferred to a base station 500 while the battery 115 is being charged. The performance information of one or more medical devices 101 can then be transferred to one or more external computing devices 340, as described herein. Similarly, in like manner system updates can be transferred from external computing devices to the medical device.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical device system, comprising:
a base station; and
a medical device including a battery having one of a processor, a persistent storage, or a communications logic, and configured to store one of a performance characteristic or system update thereon, the battery configured to be removeable from the medical device and coupled with the base station to transfer the performance characteristic from the battery to the base station, or transfer the system update from the base station to the battery.

2. The medical device system according to claim 1, wherein the medical device system is an intraosseous access system and the medical device is a driver.

3. The medical device system according to claim 2, wherein the driver is configured to place an access assembly configured to access a vasculature of a patient.

4. The medical device system according to claim 3, wherein the access assembly includes one of a needle, a needle hub, an obturator, an obturator hub, or a safety shield.

5. The medical device system according to claim 4, wherein the obturator is disposed within a lumen of the needle and is configured to prevent a tissue from entering the needle lumen.

6. The medical device system according to claim 5, wherein the safety shield is configured to couple with a distal tip of the obturator as the obturator is removed from the needle lumen, to prevent accidental needle stick injuries.

7. The medical device system according to claim 1, wherein the base station is configured to recharge the removable battery when coupled thereto.

8. The medical device system according to claim 1, wherein the base station is communicatively coupled to one of an external computing device or a network.

9. The medical device system according to claim 1, wherein a performance characteristic includes one of make, model, or serial number of one of the medical device, the battery, or an access assembly, a number of placement events performed by the medical device, a number of attachments or detachments of the access assembly, a date or time that a placement event took place, a length of time the medical device was in use, a length of time a placement event took to complete, the length of time since a performance characteristic was last transferred to the base station, a torque of a motor, a speed of the motor, a number of rotations of the motor per placement, a charge level of the battery, a number of recharging events, a length of time since the battery was last recharged, an error in the hardware, an error in the software, or an error in the operation of the medical device.

10. The medical device system according to claim 1, wherein the medical device further includes one of a second processor, a second persistent storage, or a second communications logic, configured to store a performance characteristic of the driver thereon, and configured to be coupled with one of the removable battery or the base station to transfer the performance characteristic from the medical device to one of the battery or the base station, or transfer the system update from one of the battery or the base station to the medical device.

11. The medical device system according to claim 1, wherein coupling the battery with the base station further includes one of wired communicative coupling or wireless communicative coupling, and wherein wireless communicative coupling includes one of Bluetooth, WiFi, Near Field Communication (NFC), or cellular Global System for Mobile Communication (GSM).

12. The medical device system according to claim 1, wherein one of the base station or the medical device is configured to provide an alert to indicate to a user to couple one of the battery or the medical device with the base station to transfer the performance characteristic or system update therebetween.

13. The medical device system according to claim 12, wherein the alert is one of a visual, auditory, or tactile alert provided by one of the medical device, the battery or the base station.

14. The medical device system according to claim 1, wherein the battery includes a charge level indicator.

15. A method of using a medical device system, comprising:
providing a base station, a medical device, and a removable battery configured to power the medical device, the removable battery including one of a processor, a storage, or a communications logic and configured to measure and store a performance characteristic of the medical device thereon;
activating the medical device;
measuring a performance characteristic of the medical device;
storing the performance characteristic on the removable battery;
removing the removable battery from the medical device;
coupling the removable battery with the base station; and
transferring the performance characteristic from the removable battery to the base station.

16. The method according to claim 15, wherein the medical device system is an intraosseous access system and the medical device is a driver.

17. The method according to claim 15, wherein activating the medical device includes placing an access assembly to access a vasculature of a patient.

18. The method according to claim 17, wherein the access assembly includes one of a needle, a needle hub, an obturator, an obturator hub, or a safety shield.

19. The method according to claim 18, wherein the obturator is disposed within a lumen of the needle and is configured to prevent a tissue from entering the needle lumen.

20. The method according to claim 19, further including removing the obturator from the needle lumen after the needle has access the vasculature of the patient and wherein the safety shield is configured to couple with a distal tip of the obturator as the obturator is removed from the needle lumen, to prevent accidental needle stick injuries.

21. The method according to claim 15, wherein coupling the removable battery with the base station further includes recharging the removable battery.

22. The method according to claim 15, further includes transferring the performance characteristic from the base station to one of an external computing device or a network.

23. The method according to claim 15, wherein a performance characteristic includes one of make, model, or serial number of one of the medical device, the battery, or an access assembly, a number of placement events performed by the medical device, a number of attachments or detachments of the access assembly, a date or time that a placement event took place, a length of time the medical device was in use, a length of time a placement event took to complete, the length of time since a performance characteristic was last transferred to the base station, a torque of a motor, a speed of the motor, a number of rotations of the motor per placement, a charge level of the battery, a number of recharging events, a length of time since the battery was last recharged, an error in the hardware, an error in the software, or an error in the operation of the medical device.

24. The method according to claim 15, wherein the medical device further includes one of a second processor, a second persistent storage, or a second communications logic, configured to measure and store a performance characteristic of the driver thereon.

25. The method according to claim 24, wherein the medical device is communicatively coupled with the removable battery to transfer the performance characteristic from the medical device to the battery.

26. The method according to claim 15, wherein transferring the performance characteristic further includes one of a wired communicative coupling or a wireless communicative coupling, and wherein wireless communicative coupling includes one of Bluetooth, WiFi, Near Field Communication (NFC), or cellular Global System for Mobile Communication (GSM).

27. The method according to claim 15, further including providing an alert to indicate to a user to transfer the performance characteristic from one of the removable battery or the medical device to the base station.

28. The method according to claim 27, wherein the alert is one of a visual, auditory, or tactile alert provided by one of the medical device, the removable battery or the base station.

29. A method of updating a medical device system, comprising:
providing a base station, a medical device, and a removable battery configured to power the medical device, the removable battery including one of a processor, a storage, or a communications logic and configured to store a system update thereon;
transferring the system update from an external computing device to the base station;
removing the removable battery from the medical device;
coupling the removable battery with the base station;
transferring the system update from the base station to the removable battery;
removing the removable battery from the base station;
coupling the removable battery with the medical device; and
transferring the system update from the removable battery to the medical device.

30. The method according to claim 29, wherein the medical device system is an intraosseous access system and the medical device is a driver.

31. The method according to claim 29, wherein coupling the removable battery with the base station further includes recharging the removable battery.

32. The method according to claim 29, wherein one of:
i) transferring the system update from an external computing device to the base station,
ii) transferring the system update from the base station to the removable battery, or
iii) transferring the system update from the removable battery to the medical device, includes one of a wired communicative coupling or a wireless communicative coupling, and wherein wireless communicative coupling includes one of Bluetooth, WiFi, Near Field Communication (NFC), or cellular Global System for Mobile Communication (GSM).

33. The method according to claim 29, further including providing an alert to indicate to a user to transfer the system update to the medical device.

34. The method according to claim 33, wherein the alert is one of a visual, auditory, or tactile alert provided by one of the medical device, the removable battery, or the base station.
